(19) United States
(12) Patent Application Publication
(10) Pub. No.: US 2015/0087030 A1
(43) Pub. Date: Mar. 26, 2015

(54) Pretreatment Composition for Biomass Conversion Process

(71) Applicant: Clemson University, Clemson, SC (US)

(72) Inventors: Arpan Jain, Rajasthan (IN); Terry H. Walker, Townville, SC (US)

(21) Appl. No.: 14/389,454

(22) PCT Filed: Apr. 1, 2013
(86) PCT No.: PCT/US2013/034791

§ 371 Date: Sep. 30, 2014

Related U.S. Application Data
(60) Provisional application No. 61/619,159, filed on Apr. 2, 2012.

(51) Int. Cl. D21H 17/00; D21H 17/64; C12P 7/14; C12P 19/14; C12P 19/02; D21H 17/67; D21H 17/06

(52) U.S. Cl. CPC D21H 17/74; D21H 17/675; D21H 17/64; D21H 17/06; C12P 19/14; C12P 19/02; C12P 7/14
USPC 435/99; 162/77; 162/27; 435/162; 252/186.43; 252/186.41

(57) ABSTRACT

A pretreatment composition and pretreatment process utilizing the composition is described. The pretreatment composition includes an aqueous organic solvent in combination with an alkaline component and an oxidizing agent. The pretreatment composition is useful for treating biomass as may be utilized in forming biofuel, paper, or other useful products. The biomass to be treated can include cellulosic biomass, lignocellulosic biomass, or a combination thereof.

### CROSS REFERENCE TO RELATED APPLICATION

The present application claims filing benefit of U.S. Provisional Patent Application Ser. No. 61/619,159 having a filing date of Apr. 2, 2012, which is incorporated herein by reference.

### STATEMENT REGARDING FEDERALLY SPONSORED RESEARCH

This invention was made with government support under Bioethanol Collaborative Grant no. 20-207-xxxx-0312-219-1096946 awarded by the Department of Energy. The government has certain rights in the invention.

### BACKGROUND OF THE INVENTION

Biofuels are hydrocarbons derived from biological source material (biomass) that are useful in industrial applications, for instance directly as a fuel or as raw material for the production of chemicals. Biofuels, and particularly ethanol, provide an attractive alternative to current common fuels that are derived from non-renewable resources. Presently, most of the ethanol produced from biomass is derived from corn via the conversion of corn grain starch into glucose via enzymatic hydrolysis followed by fermentation to ethanol. Unfortunately, the increased demand for corn for biofuel production has led to higher prices for all products that utilize corn in production, which includes foods that span from meat and dairy products to processed foods that incorporate corn-based products such as high fructose corn syrup.

Attempts are being made to shift the biomass source for biofuel production from crops that are used in food production such as corn and sugarcane to lignocullulosic biomass. Lignocellulosic biomass can vary greatly depending upon the specific plant, but generally includes cellulose embedded in a matrix of hemicellulose as structural material and surrounded by lignin, which serves to strengthen the cell wall and improve resistance of the plant to pests and pathogens.

Lignocellulosic biomass includes a large proportion of polysaccharides that can be converted to fermentable sugars by use of hydrolysis enzymes in biofuel production. Significant quantities of lignocellulosic biomass such as switchgrass, corn fiber, corn stover, wheat straw, rice straw, soybean residues, and the like are routinely destroyed as waste, primarily through burning, which not only wastes a good source of biofuel feedstock but also leads to environmental and health concerns. In addition, biomass such as woody biomass can be sustainably produced in many regions of the world, including the United States. Woody biomass presents good possibilities for biofuel production due to the high proportion of convertible polysaccharides. For instance, softwoods including loblolly pine have been reported to contain 45-50 wt. % cellulose, 25-35 wt. % hemicelluloses and 25-35 wt. % lignin.

Lignocellulosic biomass is currently used in the pulp and paper industry. The pretreatment necessary is very energy intensive, however, as the lignocellulosic biomass is highly recalcitrant due to natural resistance mechanisms. For instance, woody biomass such as pine wood has greater microbial recalcitrance than herbaceous biomass due to its strong structure with tightly bound high lignin content. To enhance usefulness of the lignocellulosic biomass, upstream processing including size reduction and pretreatment are a necessary step in use of lignocellulosic biomass, particularly woody biomass. In order that the production process be economically feasible, total energy consumption in the size reduction and the pretreatment steps should be minimized as much as possible.

Many methods of pretreatment currently exist for lignocellulosic biomass including physical pretreatment and chemical pretreatment. Physical pretreatment involves size reduction to increase the available surface area and enhance enzyme hydrolysis of plant polysaccharides. Chemical pretreatment methods are designed to liberate the convertible polysaccharide from the protective lignin casing as well as to reduce the crystallinity of the cellulose so as to make the polysaccharides available to the hydrolyzing microorganisms. Common chemical pretreatments include alkaline pretreatment such as ammonia soaking and recycle percolation, ammonia fiber expansion (AFEX, a physio-chemical pretreatment), organosolve processing, oxidative delignification, and supercritical explosion. In the pulp and paper industry, the pretreatment of ground biomass is carried out using either the Kraft pulping process or the sulfite process. In the Kraft pulping process, the wood chips are mixed with a white liquor that includes sodium hydroxide and sodium sulfite at 160° C.-220° C. corresponding to a steam pressure of 16 bar (absolute) for several minutes. The sulfite process uses a mixture of sulfurous acid and bisulfate ion that produces harmful gases during the process. These pretreatments have also been used in processes that include downstream enzymatic hydrolysis.

In alkaline pretreatment, lignin degradation occurs mainly due to the breakage of aryl ether linkages which constitute approximately 50-70% of total lignin linkages. However, diaryl ethers and carbon-carbon bonds of the lignin are relatively stable, and thus present barriers to complete degradation. In addition, hydroxyl ions catalyze the cleavage of ether linkages in the lignin and can result in the formation of byproducts that can inhibit microbial fermentation such as soluble sodium phenolates.

Organosolv pretreatment processing involves the use of aqueous organic solvents such as ethanol, methanol, hexane, acetone or the like and generally also utilizes inorganic acid catalysts such as hydrochloric acid (HCl) or sulfuric acid (H₂SO₄) to break the internal lignin and hemicellulose bonds. Organosolv pretreatments, as with other chemical pretreatments, often produce microbial inhibitory agents and can require large amounts of pretreatment reagents.

In order to be effective in biofuel production, a pretreatment should maximize the convertible polysaccharides released by the process while providing high process energy efficiency. What is needed in the art are improved lignocellulosic biomass chemical pretreatment processes that can more efficiently disrupt the cell wall barriers due to lignin and hemicellulose matrices as well as decrease the crystallinity of the cellulose to provide necessary access to convertible polysaccharides by hydrolytic enzymes during fermentation. Moreover, the release of convertible polysaccharides without excessive degradation of the polysaccharides due to the action of the pretreatment agents would be of benefit. In addition, a pretreatment that is cost effective through utilization of minimal amounts of reagents would be beneficial. For instance, a chemical pretreatment that presents an approach that enables recirculation of pretreatment agents to prevent excessive waste production by the process would be of benefit.

### SUMMARY

According to one embodiment, disclosed is a method for pretreating a lignocellulosic biomass. For example, the method can include combining the lignocellulosic biomass feedstock with a pretreatment composition that includes from about 50% (v/v) to about 70% (v/v) of an aqueous organic solvent, up to about 10% (w/v) of an alkaline component, and up to about 10% (v/v) of an oxidizing agent. A method can also include hydrolyzing polysaccharides of the lignocellulosic biomass feedstock to form one or more fermentable sugars.

Also disclosed is a method for forming a product such as a biofuel, pulp or paper by use of the pretreatment method. For instance, a biofuel such as ethanol can be formed by fermenting the sugars obtained from the lignocellulosic biomass feedstock.

The pretreatment composition including the aqueous organic solvent, the alkaline component, and the oxidizing agent is also described herein.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 is a flow diagram illustrating one embodiment of a process as described herein.

FIG. 2 is a flow diagram illustrating another embodiment of a process as described herein.

FIG. 3 compares the glucan yield for biomass samples after chemical pretreatment by several different chemical pretreatments followed by the same enzymatic hydrolysis treatment for all samples.

FIG. 4 compares the xylan yield for the samples of FIG. 3.

FIG. 5 compares the arabinan yield for the samples of FIG. 3.

FIG. 6 compares the glucan yield for biomass samples after chemical pretreatment by several different chemical pretreatments followed by the same enzymatic hydrolysis treatment for all samples.

FIG. 7 compares the xylan yield for the samples of FIG. 6.

FIG. 8 compares the arabinan yield for the samples of FIG. 6.

FIG. 9 illustrates the effect of potassium hydroxide (KOH) concentration in the pretreatment composition on the biomass amount remaining following pretreatment.

FIG. 10 compares the glucan yield for biomass samples after chemical pretreatment by several different chemical pretreatments followed by the same enzymatic hydrolysis treatment for all samples.

FIG. 11 compares the xylan yield for the samples of FIG. 10.

FIG. 12 compares the glucan yield for biomass samples after chemical pretreatment by several different chemical pretreatments followed by the same enzymatic hydrolysis treatment for all samples.

FIG. 13 compares the xylan yield for the samples of FIG. 112.

FIG. 14 presents the glucan yield (FIG. 14A) and the xylose yield (FIG. 14B) of a process as described herein in comparison to a different pretreatment process.

FIG. 15 illustrates a lignocellulosic biomass as may be utilized in process as disclosed herein.

FIG. 16 illustrates the biomass of FIG. 15 following combination with a pretreatment composition.

FIG. 17 illustrates a reactor that may be utilized for a pretreatment process.

FIG. 18 illustrates the pressure gauge of a reactor during a pretreatment.

FIG. 19 illustrates the solids and remaining liquid of the pretreatment composition following pretreatment.

FIG. 20 illustrates washed solids following a pretreatment process.

Other objects, features and aspects of the subject matter are disclosed in or are obvious from the following detailed description.

### DETAILED DESCRIPTION OF PREFERRED EMBODIMENTS

Reference will now be made in detail to various embodiments of the disclosed subject matter, one or more examples of which are set forth below. Each embodiment is provided by way of explanation of the subject matter, not limitation of the subject matter. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment. Thus, it is intended that the present disclosure cover such modifications and variations as come within the scope of the appended claims and their equivalents.

In general, the present disclosure is directed to a pretreatment composition and pretreatment process utilizing the composition. More specifically, the pretreatment composition includes an aqueous organic solvent in combination with an alkaline component and an oxidizing agent. The pretreatment composition can be beneficially utilized to improve release of polysaccharides from a lignocellulosic feedstock, which can then be hydrolyzed to form fermentable sugars useful for, e.g., the formation of biofuels. Other potential uses for the pretreatment product can include paper-making and enzyme production.

While not wishing to be bound to any particular theory, it is believed that the combination of the three reagents in the pretreatment composition can lead to better mass transfer in a lignocellulosic biomass during a pretreatment process that incorporates the composition. This improved mass transfer can improve release of polysaccharides from the lignin matrix as well as encourage decrease of the crystallization of the cellulose. Thus, utilization of the pretreatment composition can provide a route to increased extraction of polysaccharides from biomass while utilizing lower amounts of the reagents than has been utilized in previous pretreatment processes. In addition, it is believe that the pretreatment composition allows for improved polysaccharide extraction with less degradation of the desired convertible polysaccharides, i.e., those polysaccharides targeted in a hydrolysis process to form a fermentable sugar. Accordingly, the pretreatment composition can provide improvement to a biofuel formation process through a variety of different mechanisms.

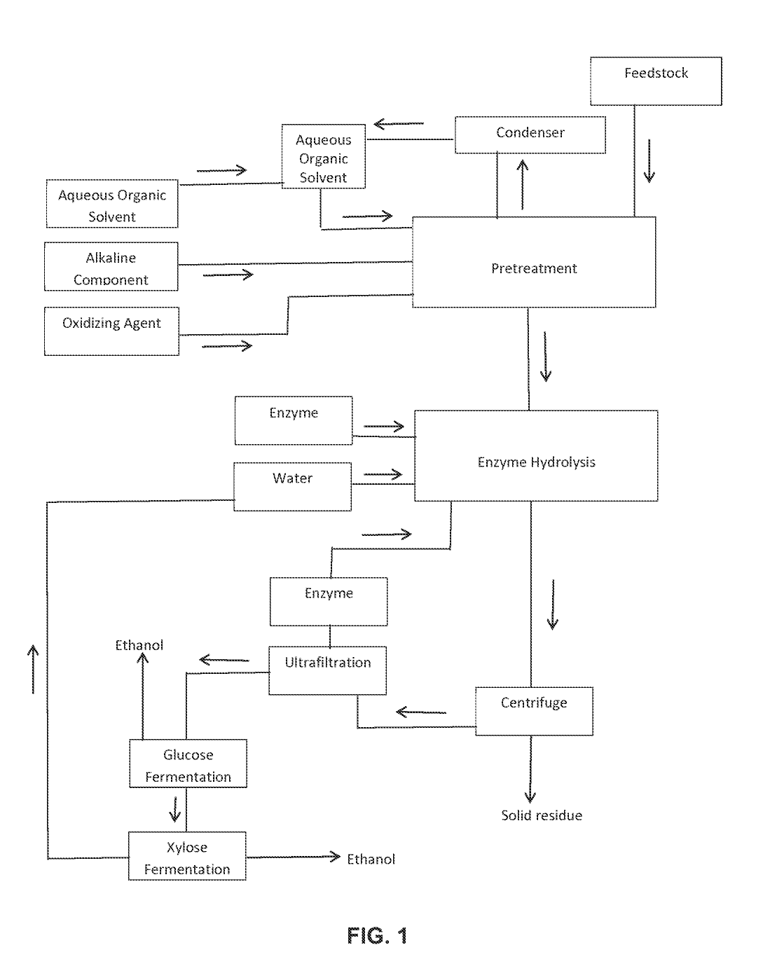

One embodiment of a biofuel formation process incorporating the pretreatment process is illustrated in the flow diagram of FIG. 1. As can be seen, the process includes the introduction of an aqueous organic solvent, an alkaline component, an oxidizing agent, and a feedstock to a pretreatment process. As illustrated in FIG. 1, the components of the pretreatment process can be added individually to the pretreatment process. Alternatively, the components may be combined and added to a pretreatment process in a preformed composition.

The aqueous organic solvent of the pretreatment composition can include lower aliphatic alcohols, lower carboxylic acids, polyhydric alcohols, or mixtures thereof. Examples of aqueous organic solvents can include, without limitation, ethanol, methanol, butanol, ethylene glycol, glycerol, 1,2-propane diol, 2,3-butanediol, acetone, formic acid, acetic acid, and the like.

The aqueous organic solvent of the pretreatment composition is believed to function as a swelling agent during pretreatment of the biomass feedstock. Swelling of the biomass feedstock can improve access of the desirable polysaccharides, e.g., the cellulose, to the hydrolysis agent in later processing of the feedstock. For instance, swelling will cause a change in dimensions of the biomass. In addition, swelling of the biomass enables the feedstock to retain its homogeneity in a microscopic environment and also encourages the biomass to become softer and more flexible, all of which can improve access of the hydrolysis agent to the targeted polysaccharides.

During pretreatment, swelling of the biomass will be facilitated through hydrogen bond formation between the aqueous organic solvent and the cellulose and/or lignin of the biomass. In addition, a strong linear correlation between activation energy of biomass swelling and the solvent molecular weight has been reported. Thus, the large molecular size of the aqueous organic solvent (for instance as compared to previously utilized swelling agents such as water) can encourage fast swelling of the biomass, particularly at elevated temperatures. For instance, when considering ethanol, the methyl group causes a subsequent increase in the activation energy of the biomass (68.3 KJ/mol) as compared to water (10.7 KJ/mol) between the temperatures of 23° C. and 60° C. However, if the aqueous organic solvent is too large, the swelling rate as well as the swelling equilibrium of a biomass sample will begin to decrease, due to the difficulty of the large molecules in diffusing into the fine capillaries of the lignocellulose matrix. Accordingly, the aqueous organic solvent of the pretreatment composition can have a number average molecular weight of between about 32 g/mol (e.g., methanol) and about 116 g/mol (e.g., heptanol).

In addition to hydrogen bonding capability and molar volume, the swelling of biomass during pretreatment can be affected by the basicity of the pretreatment composition. For instance, when considering an unground woody biomass, the swelling process will generally be more prominent in the tangential direction of the sample pieces (correlating to tangent to the direction of the annual rings of the source plant) as compared to the radial direction (from the center of the plant to peripheral edge) and will be minimal in the longitudinal direction (parallel to the plant fibers). Maximum tangential swelling can thus be correlated with the basicity of the corresponding solution.

The composition can generally include the aqueous organic solvent in an amount between about 50% (v/v) and about 70% (v/v), for instance between about 55% (v/v) and about 65% (v/v). In one embodiment, the composition can include about 60% (v/v) of the aqueous organic solvent. The relatively high concentration of the aqueous organic solvent can beneficially keep low the volatility of the alkaline component with the oxidizing agent. This can improve the safety of the process as well as provide a route to recover lignin from the feedstock biomass without the need of utilizing a lignin press.

Referring again to FIG. 1, the pretreatment composition also includes an alkaline component. The alkaline component can be a metal hydroxide or ammonium hydroxide. For instance, the alkaline component can include, without limitation, sodium hydroxide, potassium hydroxide, calcium hydroxide, magnesium hydroxide, lithium hydroxide, ammonium hydroxide, or mixtures thereof.

The alkaline component can facilitate lignin degradation through breakage of aryl ether linkages, as is known in traditional alkaline pretreatment processing techniques. This bond cleavage can increase the hydrophilicity of the lignin, which can further encourage swelling of the feedstock and increased diffusivity of the pretreatment composition throughout the feedstock as well as increased access of the hydrolysis agent to the desired polysaccharides of the biomass.

The pretreatment composition can generally include the alkaline component in an amount of up to about 10% (w/v) or up to about 5% (w/v). For instance, the composition can include the alkaline component in an amount of between about 1% (w/v) and about 5% (w/v), or in an amount of about 3% (w/v), in one embodiment. The specific concentration of the alkaline component can vary, generally depending upon the specific characteristics of the biomass as well as the process parameters (e.g., temperature, time, etc.).

Excessive degradation of the biomass polysaccharides is generally not desired during a pretreatment process. Accordingly, the pretreatment composition includes an oxidizing agent that can serve this function. Specifically, during degradation of the lignin by the alkali component, excessive degradation of the biomass polysaccharides can be prevented by oxidizing the reducing end group of the polysaccharide to an alkali-stable carboxyl group by use of the oxidizing agent.

Suitable oxidizing agents can include, without limitation, anthroquinone derivatives, polysulfides, ozone, or hydrogen peroxide. In one embodiment, hydrogen peroxide can be utilized as the oxidizing agent as it can not only stabilize the reducing end of the polysaccharides, but can also break carbon-carbon linkages and aryl-ether bonds of the lignin.

The pretreatment composition can include the oxidizing agent in an amount of up to about 10% (v/v) in one embodiment. For example, the pretreatment composition can include the oxidizing agent in an amount of between about 1% (v/v) and about 6% (v/v) or in an amount of about 5% (v/v). As with the alkaline component, the specific concentration of the oxidizing agent can vary, generally depending upon the specific characteristics of the biomass as well as the process parameters (e.g., temperature, time, etc.).

In addition to these three components, the pretreatment compositions can include optional components such as water and/or hydrogen peroxide stabilizers (e.g., sodium silicate of magnesium sulfate or similar compounds. Such stabilizers may serve to increase the efficacy of the process.

A cellulosic biomass feedstock can be added to the pretreatment process in conjunction with the pretreatment composition, as illustrated in FIG. 1. The pretreatment composition is useful for any cellulosic biomass that may be utilized in forming a biofuel. For instance, biomass feedstock that can be beneficially pretreated with the composition can include a large concentration of tightly packed lignin, such as woody feedstock as well as herbaceous biomass that comprises a lower or even no lignin content and exhibits less recalcitrance as compared to woody biomass. In one embodiment, the feedstock biomass can include a mixture of different materials, for instance a mixture of cellulosic biomass and lignocellulosic biomass.

Examples of biomass feedstock suitable for pretreatment with the composition can include, without limitation, industrial and agricultural co-products such as corn fiber, corn stover, dried distillers' grains with solubles (DDGS), wheat straw, rice straw, soybean residues, wood (including both hardwoods and softwoods), bagasse, miscanthus, reed, flax, agriculture residue such as corn husks and corn cobs, forestry residue, organic components of municipal and industrial wastes, sludge from paper manufacture, waste paper, newspaper, corrugated cardboard, waste wood (e.g., sawdust), wood chips, rice hulls, rice hulls, straw, bagasse, starch from corn, wheat oats, and barley, bark, fiberboard industry waste, bagasse pity, molasses, post-fermentation liquor, furfural still residues, aqueous oak wood extracts, oats residues, wood sugar slops, naphtha, corncob furfural residue, cotton balls, rice skin, soybean skin, soybean oil residue, cotton stems, cottonseed hulls, potatoes, sweet potatoes, lactose, sunflower seed husks, hexose sugars, pentose sugars, sucrose from sugar cane and sugar beets, corn syrup, hemp, food waste, food processing waste, and combinations of the above.

Prior to combination of the pretreatment composition with the feedstock, the feedstock can be preprocessed. For example, the feedstock can be processed according to a physical pretreatment process such as chopping, grinding, or otherwise comminuted so as to reduce the size of individual pieces of the biomass and increase the available surface area of the biomass to contact with the pretreatment composition. Due to the improved recovery available by use of the pre-treatment composition, the particle size can be relatively large, which can reduce costs of a process. By way of example, the biomass feedstock can be chopped such that individual particles of the feedstock are on the order of a few centimeters in cross section. Optionally, the biomass feedstock can be more finely ground, for instance such that the individual particles of the feedstock are on the order of a few millimeters in cross section, or less than 1 millimeter in cross section in one embodiment.

Other pretreatments as are generally known in the art can also be carried out prior to processing the feedstock with the pretreatment composition as described herein. For instance, one or more physical or physio-chemical pretreatments as are generally known in the art can be carried out prior to processing the feedstock with the pretreatment composition as described herein.

Feedstock to be processed with the pretreatment composition can have a moisture content that can vary from 0 wt. % to 100 wt. % or more. In one embodiment, a high moisture content feedstock can be utilized, for instance greater than about 50 wt. %, so as to promote better mass transport, particularly in those embodiments in which the biomass feedstock has relatively large particle sizes.

Processing with the pretreatment composition may be carried out in a batch, semi-batch or continuous operation and the ratio of pretreatment composition to feedstock can vary depending, for instance, on operation method, moisture content and/or particle size of the feedstock. In a batch operation, for example, the ratio of pretreatment composition to feedstock solids can be from about 2:1 to about 50:1, or from about 3:1 to about 15:1. In a continuous operation, i.e. with continuous supply and discharge of pretreatment composition, the liquid hourly velocity of the pretreatment composition can be in the range of from about 1 liter to about 50 liters composition per kg feed material per hour, or from about 2 L/kg/h to about 25 L/kg/h.

Processing can be carried out at various temperatures and pH and with a variety of contact times. By way of example, in one embodiment, processing can be carried out at a temperature at or slightly less than the boiling temperature of the aqueous organic solvent. For instance, when utilizing a pretreatment composition that includes ethanol as the aqueous organic solvent, the pretreatment can be carried out at a temperature of about 78° C., the atmospheric boiling temperature of ethanol. In one embodiment, the pretreatment can be carried out at a temperature between about the boiling temperature of the aqueous organic solvent and about 10° C. less than the boiling temperature of the aqueous organic solvent for instance from about 68° C. to about 78° C., or from about 65° C. to about 75° C. Of course, lower processing temperatures are also contemplated, though a lower processing temperature may preferably be utilized in a process that includes a longer contact time, so as to obtain desired results. In one embodiment, the process can be carried out at high pressure, for instance from about 180 pounds per square inch (psi) (1.24 MPa) to about 240 psi (1.65 MPa), though this is not a requirement of the pretreatment process, and higher or lower pressures can be used. For instance, the process can be carried out at atmospheric pressure, in one embodiment.

Contact time during pretreatment can vary from a few minutes to several hours, for instance from about ½ hour to about 48 hours, or from about 1 hour to about 24 hours. Likewise, pH can vary during the pretreatment, for instance from about 8 to about 13, or from about 9.5 to about 14. In the absence of one or more of the three components of the pretreatment composition, the pH can vary. For instance, a composition including ethanol and hydrogen peroxide, with no potassium hydroxide, can have a pH of between about 2 and about 3.

In addition to delignification and decrystallization of the cellulose contained in the biomass feedstock, the pretreatment process can lead to formation of a variety of organic compounds in the treated broth. For instance, succinic acid and other low molecular weight carboxylic acids can be formed during the pretreatment. Such byproducts can be have use, for instance as chemical feedstocks.

Following pretreatment, components of the pretreatment composition can be recovered and recycled. For instance, the solid and liquid fractions can be separated following suitable contact between the pretreatment composition and the biomass feedstock, and the liquid fraction can then be further separated according to standard practice to obtain useful components. For example, and as illustrated in FIG. 1, aqueous organic solvent can be recovered through a distillation and condensation process. In general, little or none of the alkaline component or the oxidizing agent will be recovered, as the oxidizing agent will be consumed during the pretreatment and the alkaline component will be held in conjunction with the dissolved solids of the feedstock.

Additional pretreatment can be carried out following processing with the disclosed pretreatment composition. For instance, other chemical pretreatment processes as are generally known in the art may be carried out following processing and prior to hydrolysis of the pretreated materials.

The pretreated lignocellulose biomass can be used in formation of a biofuel. For instance, following pretreatment(s), the pretreated biomass can be subjected to hydrolysis according to standard methodology. As illustrated in FIG. 1, an enzymatic hydrolysis utilizing a standard enzyme cocktail as is generally known in the art can be utilized according to known processing techniques to breakdown polysaccharides of the biomass to form more simple sugars such as glucan, xylan, and arabinan as well as their monomers glucose, xylose, and arabinose, that can then be fermented to form biofuel, e.g., ethanol. Following the enzyme hydrolysis, the products can be separated (as with a centrifuge as illustrated in FIG. 1), and the simple sugars can be fermented to form the biofuel, e.g., ethanol. The enzymes used in the hydrolysis process can optionally be recycled, as shown, and additional products, e.g., solid residue, can be collected. The solid residue can be used to form other useful products, such as paper as discussed above.

Of course, any hydrolysis process as is known can optionally be utilized in addition to or alternative to enzymatic hydrolysis to convert the complex plant polysaccharides of the biomass feedstock and form more simple, fermentable sugars. For instance, a chemical hydrolysis process utilizing a strong acid can alternatively be carried out.

Through utilization of the pretreatment composition, excellent recovery of polysaccharides can be obtained from the biomass feedstock. As a result, following conversion of the polysaccharides, excellent recovery of fermentable sugars can be obtained. For example more than 70%, more than 80%, or more than 90% glucan conversion can be obtained following enzyme conversion of the pretreated biomass feedstock, and more than 30%, more than 40%, or more than 50% xylan conversion of the biomass feedstock can be obtained following enzyme conversion. For instance, following pretreatment with a composition that includes about 60% ethanol, about 5% hydrogen peroxide and about 5% potassium hydroxide in which pretreatment is carried out at 78° C. (boiling point of ethanol), more than 90% glucan hydrolysis and 50% xylan hydrolysis can be obtained following conversion of the pretreated by mass with an enzyme cocktail.

Utilization of the pretreatment process is not limited to formation of biofuels, and the pretreatment composition may alternatively or additionally be utilized in other industries. For instance, the pretreatment process may be advantageously utilized in pretreatment of woody biomass in the pulp and paper industries, for instance as an alternative to the Kraft pulping process or the sulfite process. For example, disclosed pretreatment processing can be carried out in one embodiment in a time span of about 4 to about 6 hours at 70° C. in a pressure vessel (FIG. 18), which could be of great benefit in the paper and pulp industries. Moreover, the exothermic reaction of the aqueous organic solvent, the alkaline component and the oxidizing agent can increase the pressure in a reaction vessel. For instance the pressure in the reaction vessel can be from about 180 psi to about 240 psi at from about 60° C. to about 80° C. an enclosed reactor vessel. This pressure is equivalent to the processing pressure of a Kraft process at 160° C. to 240° C. In the Kraft process, the vessel is pressurized using oxygen or air to enhance delignification process. Replacement of Kraft technology with the disclosed pretreatment composition and method could be environment friendly and decrease costs.

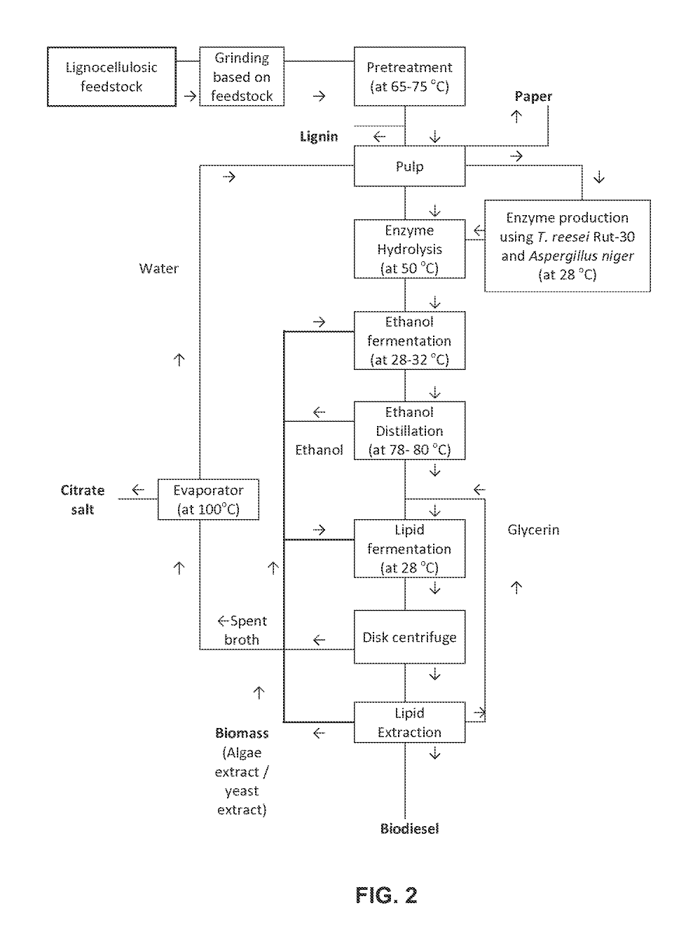

FIG. 2 illustrates an embodiment of a process utilizing the pretreatment composition that can be a combined biofuel/pulp production process. For instance, the lignocellulosic feedstock can be ground prior to the pretreatment process, as shown. Following pretreatment, the solid pulp can be separated and utilized in a paper-making process (or any other process in which the pretreated pulp may advantageously be utilized) and the remaining product can be provided to a hydrolysis process, such as the enzyme hydrolysis process utilized T. reesei and A. niger as illustrated in FIG. 2. The fermentation processes can produce ethanol, lipids, byproducts such as citrate salts, and recovery of the biomass in the production of a biofuel such as biodiesel, as shown.

The present disclosure may be better understood with reference to the Examples, provided below.

### EXAMPLES

### Materials and Methods

### Biomass Preparation

Pine wood chips including Loblolly Pine (pinus taeda) was provided by Arborgen, Inc. (Summerville, S.C.). The moisture content of unground pine wood chips was 48.0%±1.5. Before milling, the wood chips were dried at 105° C. for 2 days. The dried pine wood chips were ground using a Thomas Model 4 Wiley® Mill and sieved through 0.5-1.0 mm sieve. Moisture content of ground wood chips samples was 3.0%±0.5. FIG. 15 provides a representative image of ground, sieved woodchips used in the Examples.

### Pretreatments

The inventive pretreatment compositions tested included those formed via a combination of ethanol (EtOH), hydrogen peroxide (H₂O₂) and potassium hydroxide (KOH). 150 ml of a pretreatement composition was formed that included 90 ml of 200 proof ethanol was mixed with 21.5 ml of 35% (w/v) H₂O₂ and with a varying amount of KOH (1.5 g-7.5 g) for each sample. The formed compositions includes 60% ethanol (v/v), 5% H₂O₂ (v/v) and 1-5% KOH (w/v). A solvent to sample ratio of 10:1 (v/w) was used. FIG. 16 presents a sample of woodchips following combination with a pretreatment composition. The pretreatment process was carried at two different temperatures 38° C. and 78° C. for 24 h. A shaker flask incubator was used to maintain a stable temperature with a continuous agitation at 180 rpm. After 24 h pretreatment (FIG. 19), pretreated samples were washed with running water to remove excess composition components (FIG. 20). Carbohydrate content and digestibility were all determined following NREL Chemical Analysis and Testing Standard Procedure.

### Enzyme Hydrolysis

Pretreated samples were hydrolyzed using Accellerase® 1500 (Genecor). Initially 11±0.5 g of biomass was loaded with 50 ml of 0.1 M citric acid buffer (pH 4.7) inoculated with Accellerase 1500 enzyme (1 ml per g of biomass) along with 1 ml of 2% of sodium azide solution. Enzyme hydrolysis was controlled at temperature 50° C. and 250 rpm using a floor shaker incubator. Samples were analyzed in time interval of 0, 24, 48, and 72 hours using high performance liquid chromatography.

### Compositional Analysis

National Renewable Energy Laboratory standard method (version 07-08-2011) was followed for the determination of carbohydrate. Both pretreated and untreated pine wood chips samples were subjected to compositional analysis to determine product content. Each sample was run in triplicate. Carbohydrates were analyzed by high performance liquid chromatography (Shimadzu SIL-10AF) equipped with 356 RI detector and Bio-Rad Aminex 87H column.

Moisture content of the grinded wood chips samples were determined by oven-drying method at 105° C. to constant weight.

Statistical analysis was carried out using SAS (statistical analysis system) program (SAS for Windows; version 9.1, Cary, N.C.). Paired t-tests and probability level 0.05 (p-value=0.05) were applied to test the statistical significance of treatment variables.

### Example 1

Ground pine wood chips ranging from about 0.5 mm to about 1.0 mm in size) were analyzed under five different pretreatments, as described in Table 1 (all amounts of KOH are provided as w/v %, and all values of H₂O₂ and EtOH are provided as v/v %):

TABLE 1

| Sample | |
|---|---|
| No. | Pretreatment Formulation and Process |
| 1 | 5% KOH + 5% H₂O₂ @ 80° C. for 24 h |
| 2 | 5% KOH + 5% H₂O₂ @ 80° C. for 6 h + @120° C. for 18 h |
| 3 | 5% KOH + 5% H₂O₂ + 60% EtOH @ 80° C. for 24 h |
| 4 | 5% H₂O₂ @ 80° C. for 24 h |
| 5 | 5% KOH @ 120° C. for 24 h |

Following pretreatment, the biomass was subjected to either an enzyme hydrolysis process or a sulfuric acid hydrolysis process. The enzyme hydrolysis process was performed using Accellerase® 1500 (Genecor). Initially 11 g of biomass was loaded with 50 ml of 0.1 M citric acid buffer (pH 4.7) inoculated with Accellerase® 1500 enzyme (1 ml per g of biomass) along with 1 ml of 2% of sodium azide solution. Enzyme hydrolysis was controlled at temperature 50° C. and 250 rpm using a floor shaker incubator. Samples were analyzed at time intervals of 0, 24, 48, and 72 hours using high performance liquid chromatography.

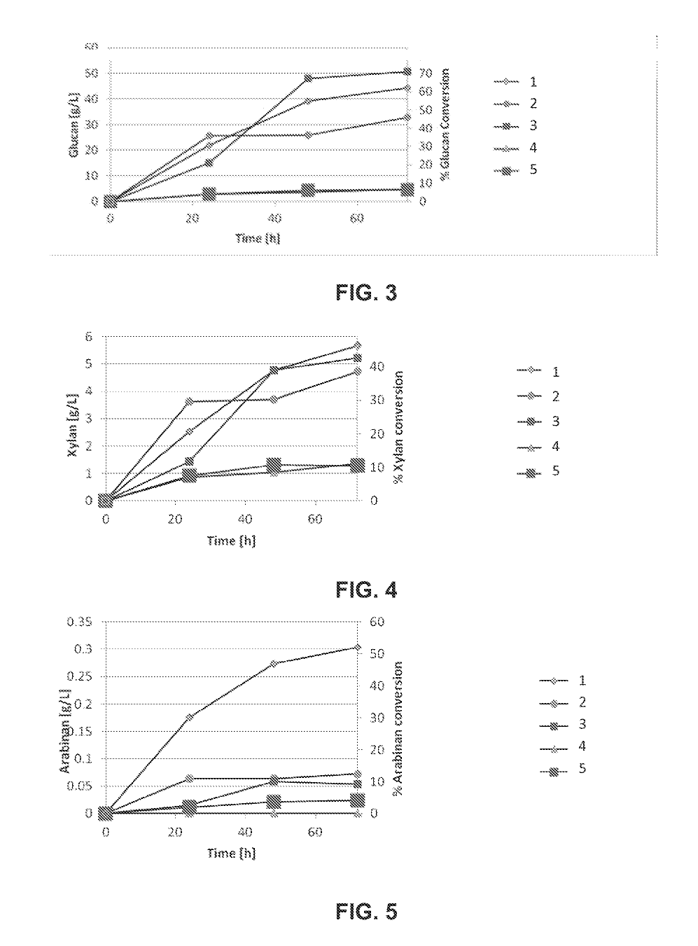

Results for the amount of glucan, xylan, and arabinan yield via the enzyme hydrolysis process are shown in FIG. 3, FIG. 4 and FIG. 5, respectively. As can be seen with reference to the Figures, pretreatment using 5% KOH and 5% H₂O₂ @ 80° C. for 24 h (sample 1) and 5% KOH+5% H₂O₂+60% EtOH @ 80° C. for 24 h (sample 3) followed by enzyme hydrolysis were found effective in terms of sugar conversion (FIG. 1, FIG. 2 and FIG. 3), with the inventive pretreatment sample 3 providing better results. The pretreatment using sample 3 resulted in 70% glucan conversion and 43% xylan conversion. The yield using sample 1 was found to be 60% glucan conversion and 35% xylan conversion of the pretreated biomass (biomass lost in the washed stream was not included).

According to the sulfuric acid hydrolysis method, 150 mg of sample was taken in a pressure tube. About 1.5 ml of 70% sulfuric acid was added to the tube. The pressure tube was incubated on a heater block at 36° C. for 1 hour. The mixture was stirred every 10 minutes using a glass rod. Following, 42 ml of deionized water was added to the tube and the contents were transferred to an autoclave at 121° C. for 1 hour. The sulfuric acid hydrolysate was neutralized to pH 6-7 using calcium carbonate. Results are shown in Table 2, below. Values of the glucan, xylan, and arabinan are provided as a percentage of the total amount of carbohydrate in the sample.

TABLE 2

| Sample No. | Glucan (wt %) | Xylan (wt %) | Arabinan (wt %) |
|---|---|---|---|
| Without | 52.22 | 21.48 | 1.22 |
| pretreatment | | | |
| 1 | 57.30 | 12.23 | 0.44 |
| 2 | 62.64 | 11.30 | 0.83 |
| 3 | 62.08 | 10.51 | 0.68 |
| 4 | 52.07 | 14.95 | 0.54 |
| 5 | 53.02 | 15.05 | 0.61 |

### Example 2

Unground pine wood chips approximately 30 mm by 25 mm by 4 mm in size were analyzed under five different pretreatments as described in Table 3, below:

TABLE 3

| Sample | |
|---|---|
| No. | Pretreatment Formulation and Process |
| 6 | 60% EtOH + 5% H₂O₂ @ 78° for 24 h |
| 7 | 60% EtOH + 10% H₂O₂ @ 78° C. for 24 h |
| 8 | 60% EtOH + 5% KOH + 10% H₂O₂ @ 78° C. for 24 h |
| 9 | 60% EtOH + 5% KOH + 5% H₂O₂ @ 78° C. for 48 h |
| 10 | 60% EtOH + 5% KOH + 2% H₂O₂ @ 78° C. for 48 h |

Enzyme hydrolysis was performed using Accellerase® 1500 (Genecor). Initially 11 g of biomass was loaded with 50 ml of 0.1 M citric acid buffer (pH 4.7) inoculated with Accellerase® 1500 enzyme (1 ml per g of biomass) along with 1 ml of 2% of sodium azide solution. Enzyme hydrolysis was controlled at a temperature of 50° C. and 250 rpm using floor shaker incubator. Samples were analyzed in time interval of 0, 24, 48, and 72 hours using high performance liquid chromatography.

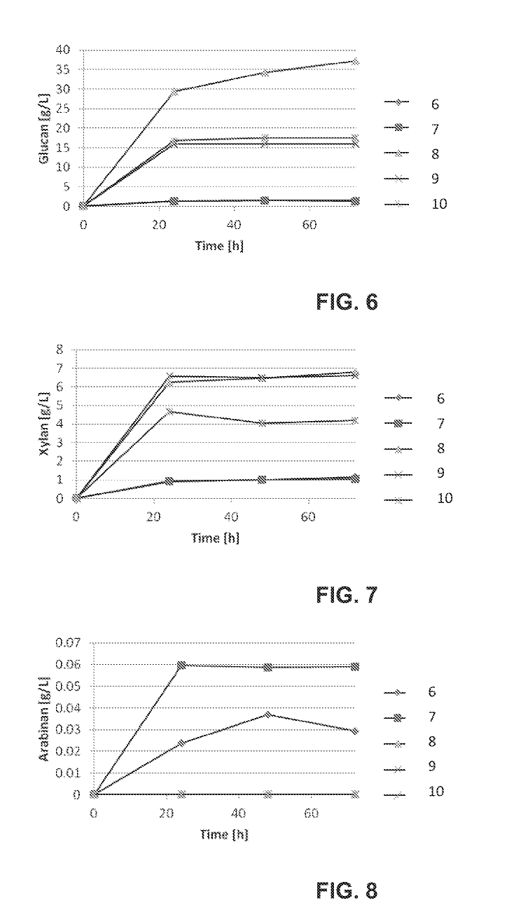

Results for the amount of glucan, xylan, and arabinan yield via the enzyme hydrolysis process are shown in FIG. 6, FIG. 7, and FIG. 8, respectively. As can be seen, pretreatment using sample 8 was effective in terms of sugar conversion.

### Example 3

Several different pretreatment compositions were formed as described in Table 4, below. The pretreatment process included inoculation of 15 g of ground loblolly pine wood chips with 150 ml of the different pretreatment compositions 11-16 as described in Table 4. The ratio of 1:10 (biomass to pretreatment composition) was used. Pretreatment for each composition was carried out at 78° C. for a period of 24 hrs.

TABLE 4

| Sample No. | Pretreatment Formulation |
|---|---|
| 11 | 60% Ethanol + 5% H₂O₂ |
| 12 | 60% Ethanol + 5% H₂O₂ + 1% KOH |
| 13 | 60% Ethanol + 5% H₂O₂ + 2% KOH |
| 14 | 60% Ethanol + 5% H₂O₂ + 3% KOH |
| 15 | 60% Ethanol + 5% H₂O₂ + 4% KOH |
| 16 | 60% Ethanol + 5% H₂O₂ + 5% KOH |

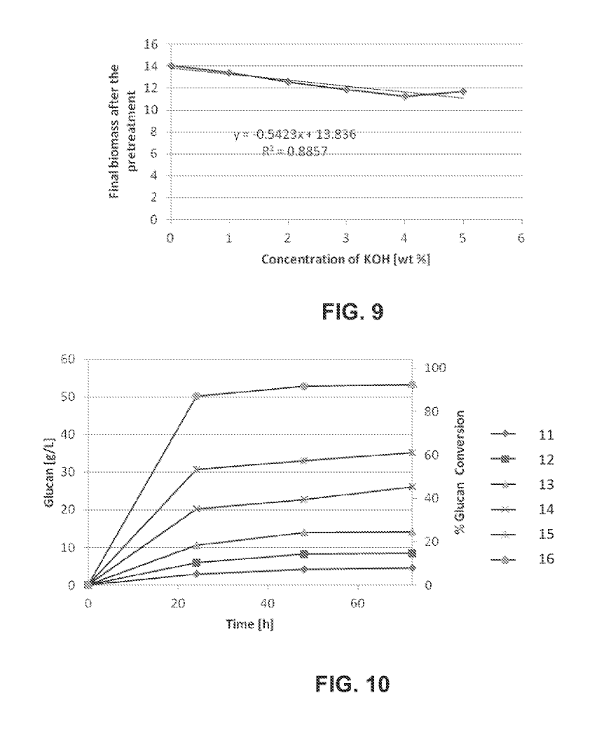

The loss of feedstock weight was monitored after each of the pretreatments. Table 5 and FIG. 9 present the results of this monitoring. As can be seen, biomass concentration decreases with increasing KOH concentration in the pretreatment composition.

TABLE 5

| | Initial Mass | Final mass after | Mass lost during |
|---|---|---|---|
| Pretreatment | loading (g) | pretreatment (g) | pretreatment (%) |
| 11 | 15 | 14.05 | 6.33 |
| 12 | 15 | 13.42 | 10.53 |
| 13 | 15 | 12.58 | 16.13 |
| 14 | 15 | 11.87 | 20.87 |
| 15 | 15 | 11.28 | 24.8 |
| 16 | 15 | 11.68 | 22.13 |

Initial pH and final pH were also measured before and after the pretreatments. Results are shown in Table 6.

TABLE 6

| Pretreatment | Initial pH reading | Final pH reading |
|---|---|---|
| 11 | 3.47 | 2.10 |
| 12 | 10.53 | 6.37 |
| 13 | 11.25 | 10.41 |
| 14 | 11.65 | 15.01 |
| 15 | 11.93 | 14.92 |
| 16 | 12.29 | 13.49 |

Following pretreatment, the samples were subjected to a sulfuric acid hydrolysis process as described above. Table 7, below, provides the composition of tested sugars in the biomass following the sulfuric acid hydrolysis.

TABLE 7

| | Glucan [wt %] | | Xylan [wt %] | | Arabinan [wt %] | |
|---|---|---|---|---|---|---|
| Sample No. | Mean | Std dev. | Mean | Std dev. | Mean | Std dev. |
| Without | 52.22 | 0.24 | 21.48 | 0.24 | 1.06 | 0.04 |
| Pretreatment | | | | | | |
| 11 | 44.53 | 0.38 | 17.55 | 0.19 | 1.30 | 0.20 |
| 12 | 46.88 | 0.14 | 16.42 | 0.60 | 1.44 | 0.67 |
| 13 | 49.81 | 1.18 | 15.76 | 0.37 | 2.14 | 0.06 |
| 14 | 51.28 | 2.27 | 14.37 | 0.76 | 1.60 | 0.76 |
| 15 | 52.52 | 1.78 | 14.07 | 0.74 | 1.56 | 0.71 |
| 16 | 51.92 | 2.42 | 14.12 | 0.47 | 1.13 | 0.72 |

As can be seen, glucan content of the treated biomass was found to increase with increasing KOH concentration in the pretreatment composition. The glucan yield using sample nos. 15 and 16 for pretreatments were not significantly different. However, a significant difference was observed in the yields of xylan and arabinan content in sample nos. 15 and 16 as shown in the table above (Table 7). Xylan content, however, decreased with increasing KOH concentration in the pretreatment solution. Arabinan content increased to sample no. 13 and then began to decrease with further increase in KOH concentration. As can be seen, the concentration of KOH in the pretreatment composition plays an important role in the recovery of different sugars.

Enzyme hydrolysis of biomass pretreated with pretreatment composition sample nos. 11-16 was performed using Accellerase® 1500 (Genecor). Initially 11 g of biomass was loaded with 50 ml of 0.1 M citric acid buffer (pH 4.7) inoculated with Accellerase® 1500 enzyme (1 ml per g of biomass) along with 1 ml of 2% of sodium azide solution. Enzyme hydrolysis was controlled at a temperature of 50° C. and 250 rpm using floor shaker incubator. Samples were analyzed in time interval of 0, 24, 48, and 72 hours using high performance liquid chromatography.

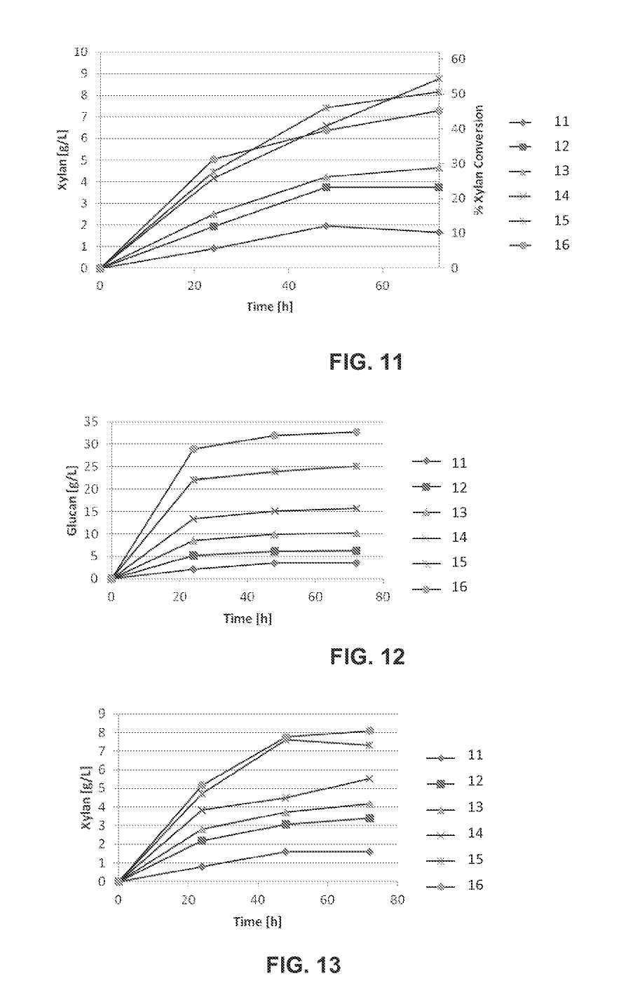

Results for the amount of glucan and xylan yield via the enzyme hydrolysis process are shown in FIG. 10 and FIG. 11, respectively.

### Example 4

Pretreatment solution nos. 11-16, described in Table 7 were prepared and pretreatment of ground loblolly pine was carried out at 38° C. for a period of 24 hrs. Following pretreatment, enzyme hydrolysis by use of Accellerase® was carried out as described above. Results for the amount of glucan and xylan yield via the enzyme hydrolysis process are shown in FIG. 12 and FIG. 13, respectively.

### Example 5

Pine woods chips were received from Arbogen. The moisture content of the received pine wood chips was measured at 10%. The pine wood chips were oven dried for 3 days at 60° C. The dried chips were ground by a Wiley mill (Thomas Model 4 Wiley® Mill) and sieved with 0.5 mm mash size. Milling and sieving resulted in densification of the biomass.

### Ethyl-Hydro-Oxides Pretreatment

Ethyl-hydro-oxide (EHO) pretreatment consisted of 60% (v/v) ethanol, 5% (w/v) potassium hydroxide and 5% (v/v) hydrogen peroxide at 78° C. for 24 h.

### Alkaline Peroxide Pretreatment

Alkaline-hydroxide pretreatment consisted of 5% (w/v) potassium hydroxide and 5% (v/v) hydrogen peroxide in water at 78° C. for 24 h.

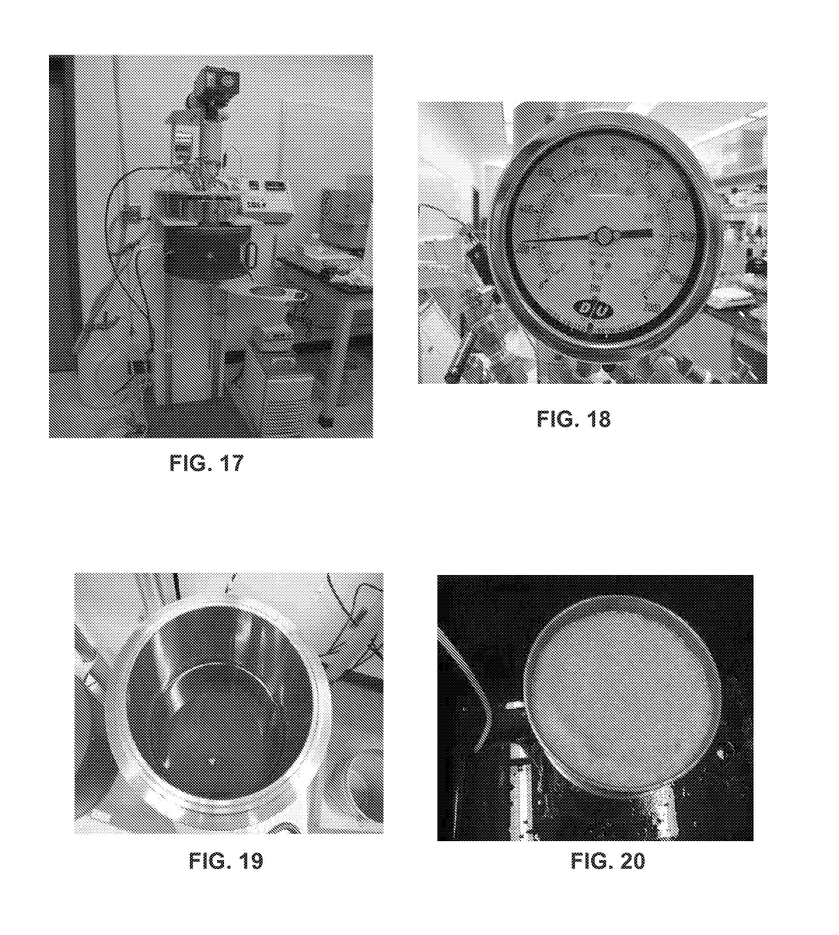

Initially, 8.0±0.1 g of pine wood chips (0.5-1.0 mm) were added with the above solvents in the ratio of 1:10 (w/v) in 500 ml bottles with a screw cap (Gupta 2008) and pretreatment was carried out in a Parr reactor as illustrated in FIG. 17. During pretreatment, the pressure in the reactor was monitored (FIG. 18)

Both alkaline-peroxide-treated solids and EHO-treated solids were hydrolysed using Accellerase® 1500. In 250 mL stopper conical flasks pretreated pine wood solids were added with 25 ml of 0.1 M citric acid buffer (pH 4.8), 80 FPU of Accellerase 1500 enzyme per gram of biomass, which was supplemented with 2.5 ml of 2% (w/v) sodium azide solution to prevent microbial contamination. The flasks were incubated at 50° C. in a floor shaker incubator (New Brunswick Scientific) at 250 rpm. Samples of 1.5 ml were taken from the flask in time intervals of 24, 48 and 72 h.

NREL standard methods (2008) were followed for the determination of carbohydrate and lignin content of loblolly pine wood chips before and after the pretreatments. Each sample was run in triplicate. Carbohydrates were analyzed by HPLC (Shimadzu SIL-10AF and 355 RI detectors, Palo Alto, Calif.) equipped with a Bio-Rad Aminex 87H column.

Moisture content of the pinewood chips was determined by oven-drying method at 105° C. to constant weight.

The effects of the EHO and alkaline-peroxide pretreatments on the composition of grinded pine wood chips are shown in Table 8 and Table 9, below. As can be seen, EHO-treated solids and alkaline-peroxide treated solids resulted in 20 to 25 wt. % loss from the initial weight of the sample (Table 8). In addition, the results indicated an increase in glucan content and decrease in xylan content over the period of 24 hours pretreatment (Table 9). EHO-treated resulted in a relative increase of 37 wt. % glucan and 18.7 wt. % arabinan to alkaline-peroxide-treated solids. However, alkaline-peroxide-treated solids retained 7.8 wt. % xylan relative to EHOs-treated solids (Table 9). The solid residue (lignin+ash) was found 72 wt. % and 36.7 wt. % respectively, lower for EHO-treated solids and alkaline-peroxide-treated solids compared to control (water-treated solids, Table 9).

TABLE 8

| | Pretreatment | | |
|---|---|---|---|
| | Water | EHOs | NaOH + H₂O₂ |
| | Ratio | | |
| | 1:10 | 1:10 | 1:10 |
| | Temperature | | |
| | 78° C. | 78° C. | 78° C. |
| Before pretreatment (g) | 8.00 ± 0.1 | 8.00 ± 0.1 | 8.00 ± 0.1 |
| After pretreatment (g) | 7.93 ± 0.1 | 6.02 ± 0.33 | 6.20 ± 0.2 |

TABLE 9

| | Pretreatment | | |
|---|---|---|---|
| | Water | EHOs | NaOH + H₂O₂ |
| | Ratio | | |
| | 1:10 | 1:10 | 1:10 |
| | Temperature | | |
| | 78° C. | 78° C. | 78° C. |
| | Processing time | | |
| | 24 h | 24 h | 24 h |
| Component | [wt. %] | [wt. %] | [wt. %] |
| Glucan | 44.3 ± 0.5 | 62.1 ± 0.5 | 57.3 ± 0.4 |
| Xylan | 24.6 ± 1.7 | 10.5 ± 0.4 | 12.2 ± 0.2 |
| Arabinan | 2.0 ± 0.1 | 0.7 ± 0.1 | 0.4 ± 0.1 |
| Solid residues | 29.6 ± 1.8 | 8.8 ± 1.9 | 20.0 ± 2.2 |
| (Lignin + Ash) | | | |

The ethanol concentration was measured before and after the EHO pretreatment. In the presence of alkaline and strong oxidizing agents, the initial concentration of ethanol was not changed during the pretreatment.

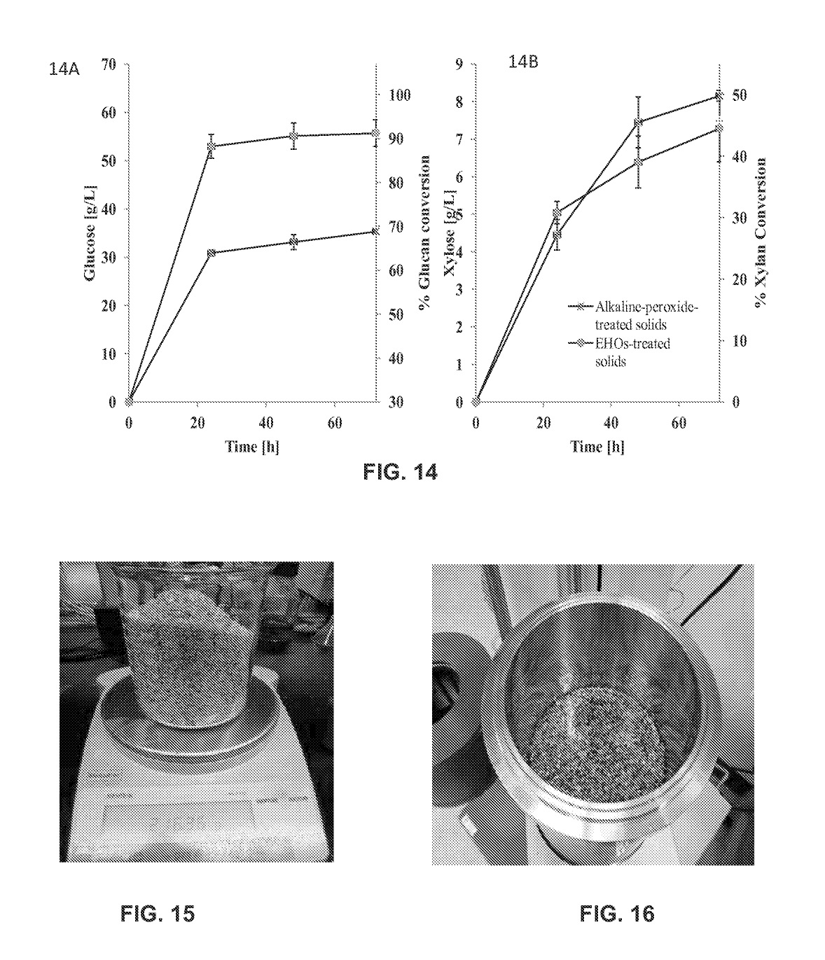

Enzyme hydrolysis of EHO-treated and alkaline-oxide-treated solids resulted in 90/45% and 70/50% of glucan/xylan yields respectively at 72 hours (FIG. 14). A significant difference was found in enzyme hydrolysis yields of EHO-treated solids and alkaline-peroxide-treated solids.

As can be seen, the combined action of the organic solvent along with KOH and H₂O₂ at 78° C. resulted in the higher saccharification yield of softwood. The activation energy of wood swelling lies in the range of 75 KJ mol⁻¹-89 KJ mol⁻¹. It is understood that the addition of the —CH₃ group causes a subsequent increase in the activation energy of wood using ethanol (68.3 KJ mol⁻¹) compared to water (10.7 KJ/mol) between the temperature of 23° C.-60° C. The maximum swelling of wood in organic solvent is accompanied by the solvent basicity, the molar volume and the hydrogen bonding capability. Thus, the addition of 60% (v/v) ethanol in the EHO-solvent is understood to improve removal of the lignin and provides a route for the KOH to swell cellulose fibers.

In addition, the produced reactive species of H₂O₂ (hydroxyl radicals, superoxide anion, and hydroperoxide anion) may enhance the breakage of diaryl ethers and carbon-carbon bonds along with aryl-ether linkages in the presence of ethanol at 78° C. An advantage of the disclosed pretreatment process includes increasing the solubility of lignin and swelling (with decreasing crystallinity) of softwood at 78° C.

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure which is defined in any following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present disclosure.

1. A method for treating a lignocellulosic biomass comprising:
combining a lignocellulosic biomass feedstock with a pretreatment composition, the pretreatment composition including from about 50% (v/v) to about 70% (v/v) of an aqueous organic solvent, up to about 10% (w/v) of an alkaline component, and up to about 10% (v/v) of an oxidizing agent.

2. The method according to claim 1, further comprising hydrolyzing polysaccharides of the lignocellulosic biomass feedstock subsequent to the combining step to form one or more fermentable sugars.

3. The method according to claim 1, wherein the aqueous organic solvent is an aliphatic alcohol, a carboxylic acid, a polyhydric alcohol, or mixtures thereof.

4. The method according to claim 1 further comprising comminuting the lignocellulosic biomass feedstock.

5. The method according to claim 1, wherein the ratio of the pretreatment composition to the lignocellulosic biomass feedstock is from about 2:1 to about 50:1.

6. The method according to claim 1, wherein the liquid hourly velocity of the pretreatment composition in the combining step is from about 1 liter to about 50 liters pretreatment composition per kilogram lignocellulosic biomass feedstock per hour.

7. The method according to claim 1, wherein the combining step is carried out at a temperature that is between about the boiling temperature of the aqueous organic solvent and about 10° C. less than the boiling temperature of the aqueous organic solvent.

8. The method according to claim 1, wherein the combining step is carried out for a period of time from about ½ hour to about 48 hour and/or at a pH of from about 8 to about 13.

9. A method for forming a biofuel comprising treating a lignocellulosic biomass according to the method of claim 2 and fermenting the one or more fermentable sugars to form the biofuel.

10. The method according to claim 9, wherein the biofuel is ethanol or diesel.

11. A method for forming pulp or paper comprising treating a lignocellulosic biomass according to the method of claim 1, wherein the lignocellulosic biomass comprises woody biomass.

12. A pretreatment composition for a lignocellulosic biomass comprising from about 50% (v/v) to about 70% (v/v) of an aqueous organic solvent, up to about 10% (w/v) of an alkaline component, and up to about 10% (v/v) of an oxidizing agent.

13. The pretreatment composition of claim 12, wherein the aqueous organic solvent has a number average molecular weight of between about 32 grams per mole and about 116 grams per mole.

14. The pretreatment composition of claim 12, wherein the aqueous organic solvent is an aliphatic alcohol, a carboxylic acid, a polyhydric alcohol, or mixtures thereof.

15. The pretreatment composition of claim 12, wherein the alkaline component is a metal hydroxide or ammonium hydroxide or mixtures thereof.

16. The pretreatment composition of claim 12, wherein the oxidizing agent is an anthraquinone derivative, a polysulfide, ozone, or hydrogen peroxide.

17. The pretreatment composition of claim 12, wherein the pretreatment composition further includes water.

18. The pretreatment composition of claim 12, wherein the oxidizing agent is hydrogen peroxide and the pretreatment composition further includes a hydrogen peroxide stabilizer such as sodium silicate of magnesium sulfate.

19. The pretreatment composition of claim 12, wherein the pretreatment composition includes from about 1%(w/v) to about 5% (w/v) of the alkaline component.

20. The pretreatment composition of claim 12, wherein the pretreatment composition includes from about 1% (v/v) to about 6% (v/v) of the oxidizing agent.

21. The method according to claim 2, wherein the polysaccharides are hydrolyzed according to an enzymatic hydrolysis process or a strong acid hydrolysis process.

22. The method according to claim 3, wherein the aqueous organic solvent has a number average molecular weight of between about 32 grams per mole and about 116 grams per mole.

23. The method according to claim 1, wherein the ratio of the pretreatment composition to the lignocellulosic biomass feedstock is from about 3:1 to about 15:1.

24. The method according to claim 1, wherein the liquid hourly velocity of the pretreatment composition in the combining step is from about 2 liters to about 25 liters pretreatment composition per kilogram lignocellulosic biomass feedstock per hour.

25. The pretreatment composition of claim 12, wherein the aqueous organic solvent is ethanol, butanol, methanol, ethylene glycol, glycerol, 1,2-propane dial, 2,3-butanediol, acetone, formic acid, acetic acid, or mixtures thereof.

26. The pretreatment composition of claim 12, wherein the alkaline component is sodium hydroxide, potassium hydroxide, calcium hydroxide, magnesium hydroxide, lithium hydroxide, ammonium hydroxide, or mixtures thereof.
